(19) United States
(12) Patent Application Publication
(10) Pub. No.: US 2025/0382600 A1
(43) Pub. Date: Dec. 18, 2025

(54) ALPHA-AMYLASE MUTANT AND USE THEREOF

(71) Applicant: GUANGDONG VTR BIO-TECH CO., LTD., Zhuhai (CN)

(72) Inventors: Xing Jin, Zhuhai (CN); Yangyuan Li, Zhuhai (CN); Yupeng Wu, Zhuhai (CN); Zhiliang Zhang, Zhuhai (CN); Qi Wu, Zhuhai (CN); Mingxi Li, Zhuhai (CN)

(21) Appl. No.: 18/914,076

(22) Filed: Oct. 12, 2024

Related U.S. Application Data
(63) Continuation of application No. PCT/CN2024/099098, filed on Jun. 14, 2024.

(51) Int. Cl. C12N 9/28; C12N 1/20; C12N 15/70; C12N 15/75; C12P 7/06; C12R 1/07; C12R 1/19

(52) U.S. Cl. CPC C12N 9/2417; C12N 1/20; C12N 15/70; C12N 15/75; C12P 7/065; C12R 2001/07; C12R 2001/19; C12Y 302/01001

(57) ABSTRACT

Discloses an α-amylase mutant and a use thereof. Compared with a parental α-amylase with an amino acid sequence shown in SEQ ID NO: 1, the α-amylase mutant has a disulfide bond site pair mutation, the disulfide bond site pair has the following characteristics: i. the disulfide bond site pair is not within 5Å range of an active site of the α-amylase mutant; ii. a difference value in amino acid site numbers is greater than 10; iii. a distance between SG atoms of two cysteines forming a disulfide bond is within 5Å; and iv. a Chi3 angle is 60°<Chi3<120° or −60°>Chi3>−120°. The heat resistance and/or acid resistance of the α-amylase mutants provided in the present disclosure have been significantly improved.

### CROSS-REFERENCE TO RELATED APPLICATIONS

The present application is a continuation application of PCT application No. PCT/CN2024/099098 filed on Jun. 14, 2024, the contents of which are incorporated by reference herein in their entirety.

### REFERENCE TO SEQUENCE LISTING

This application includes a Sequence Listing filed electronically as an XML file named “Sequence listing_ERICL-24007-USPT.xml”, created on Oct. 10, 2024, with a size of 3,827 bytes. The Sequence Listing is incorporated herein by reference.

### TECHNICAL FIELD

The present disclosure belongs to the technical fields of genetic engineering and enzyme engineering, and specifically relates to an α-amylase mutant and a use thereof.

### BACKGROUND

Enzyme molecular modification can be achieved through rational design of proteins or directed evolution technology. By modifying the molecules of natural enzymes, new enzymes with higher stability, higher activity, higher selectivity, and higher tolerance to extreme environments can be provided for industrial production. Rational design is one of the important methods in protein engineering. Based on a clear understanding of the structure, function, and molecular mechanisms related to the properties of proteins, changes in specific amino acid sites in protein molecules are first theoretically designed to obtain some mutants with special properties. The difficulty of this research lies in how to find effective modification sites.

At present, the number of protein sequences stored in the three major databases of Genbank, EMBL, and DDBJ is growing exponentially, while the growth rate of the number of protein three-dimensional structural information recorded in the Protein Data Bank (PDB) database lags far behind. Given the extreme difficulty in obtaining protein structures through experimental methods, homology modeling technology has become a commonly used bioinformatics method for modern biologists to obtain protein three-dimensional structures. Currently, commonly used protein homology modeling programs include Swiss-Model, CPHmodel, SDSC1, 3D-jigsaw, InsightII, sybyl, COMPOSER, Modeller, AlphaFold2, and so on.

With the increasing maturity of molecular simulation technology after the determination of protein three-dimensional structures, it provides strong support for correlating three-dimensional structures with protein functions. Molecular simulation technology can be used to simulate various dynamic behaviors of molecules, glassy molecular structures, characteristics of molecular motion, protein folding and unfolding, and so on. Common molecular force fields include Amber (Assisted Model Building with Energy Refinement) and Charmm (Chemistry at HARvard Molecular Mechanics).

Amber is used for computational simulations of biomacromolecules such as proteins, nucleic acids, and sugars. AMBER offers two main components: a set of molecular mechanics force fields for simulating biomolecules, and a program for molecular simulations, including source code and demonstrations.

CHARMM is a widely recognized and applied molecular simulation program for the simulation of biomacromolecules, encompassing molecular dynamics, energy minimization, Monte Carlo simulations, and so on. The CHARMM force field employed by the program can provide users with empirical energy calculations for various small molecules and macromolecules (including proteins, nucleic acids, and sugars), such as thermodynamic free energy and folding free energy.

α-amylase (α-1,4-glucan-4-glucanohydrolase, E.C.3.2.1.1) constitutes a group of enzymes that catalyze the hydrolysis of starch, as well as other linear and branched 1,4-glycosidic oligosaccharides and polysaccharides. α-amylase can be applied to the initial stage of starch processing (liquefaction), wet corn milling, alcohol production, used as a cleaner in detergent matrices, used for starch desizing, used in baking and beverage industries, used in drilling processes in oil mines, used in the deinking process of recycled paper, and used in animal feed.

Although some successes have been achieved in the above application fields with the currently available α-amylase, in recent years, with the changes in processing conditions in the starch raw material processing industry and alcohol industry, the enzyme preparation industry has been required to continuously update and improve the types of enzymes to meet industrial needs. For example, in the process of starch saccharification industry, generally, amylase is first used to liquefy starch, and then glucoamylase is added for saccharification to produce glucose.

However, the currently available amylases have a suitable pH range of 6.0 to 6.5, with an optimal pH of 6.0, and they deactivate below a pH of 5.0, while the optimal pH for the saccharification step is around 4.5. Additionally, the widely used jet liquefaction process in sugar production has a jet temperature of generally 105 to 108° C., with a dwell time of 5 to 6 minutes in the intermediate high-temperature holding tank. Therefore, after completing the starch liquefaction step, the pH and temperature need to be repeatedly adjusted before adding glucoamylase, leading to complexity in production and environmental issues.

In traditional liquor production, due to incomplete solid-state fermentation, the distiller's grains commonly contain more than 10% residual starch, and the pH in the distiller's grains is very low. When the distiller's grains are returned to the fermentation pit, the acidic pH environment is not suitable for the action of α-amylase.

In summary, the current α-amylase still fails to meet the requirements of the starch raw material processing industry for high temperature resistance, acid resistance, and high activity, and cannot adapt well to the application in starch-based deep processing industries such as sugar production, brewing, alcohol production, and organic acid production. Therefore, using genetic engineering to modify α-amylase and develop α-amylase with further improved high temperature and/or acid resistance is of great significance for starch-based deep processing industries.

### SUMMARY

The present disclosure aims to solve at least one of the technical problems existing in the above-mentioned prior art. To this end, the present disclosure proposes an α-amylase mutant and a use thereof.

The present disclosure aims to provide an α-amylase mutant that is resistant to high temperatures and/or acids. Using a computer-aided approach, the present disclosure predicts the formation of disulfide bond sites within the amylase structure based on the spatial structure and functional requirements of the parental α-amylase. Subsequently, the influence of amino acid site mutations capable of forming disulfide bonds on the formation of hydrogen bonds and salt bridges within the enzyme molecule is analyzed, determining the site where disulfide bonds should be introduced into the amylase protein structure. The α-amylase mutant provided by the present disclosure exhibits excellent high-temperature resistance and/or acid resistance, thereby better meeting the needs of starch-based deep processing industries such as sugar making, brewing, alcohol production, and organic acid production.

The present disclosure provides an α-amylase mutant.

Specifically, the α-amylase mutant has α-amylase activity. Compared with a parental α-amylase with an amino acid sequence shown in SEQ ID NO: 1, the α-amylase mutant has a disulfide bond site pair mutation, and the disulfide bond site pair has the following characteristics:
i. the disulfide bond site pair is not within 5 Å range of an active site of the α-amylase mutant;
ii. a difference value in amino acid site numbers is greater than 10;
iii. a distance between SG atoms of two cysteines forming a disulfide bond is within 5 Å; and
iv. a Chi3 angle is 60°<Chi3<120° or −60°>Chi3>−120°.

SEQ ID NO: 1:
AAPFNGTMMQYFEWYLPDDGTLWTKVANEANNLSSLGINALWLPPAYKG
TSRSDVGYGVYDLYDLGEFNQKGTVRTKYGTKAQYLQAIQAAHAAGMQV
YADVVFDHKGGADGTEWVDAVEVNPSDRNQEISGTYQIQAWTKFDFPGR
GNTYSSFKWRWDHFDGVDWDESRKLSRIYKFRGKAWDWEVDTEFGNYDY
LMYADLDMDHPEVVTELKNWGKWYVNTTNIDGFRLDAVKHIKFQFFPDW
LSYVRSQTGKPLFTVGEYWSYDINKLHNYITKTDGTMSLFDAPLHNKFY
TASKSGGAFDMRTLMTNTLMKDQPHLAVTFVDNHDTEPGQALQSWVDPW
FKPLAYAFILTRQEGYPCVFYGDYYGIPQYNIPSLKSKIDPLLIARRDY
AYGTQHDYLDHSDIIGWTREGVTEKPGSGLAALITDGPGGSKWMYVGKQ
HAGKVFYDLTGNRSDTVTINSDGWGEFKVNGGSVSIYVQR.

The α-amylase mutant has improved characteristics compared with the parental α-amylase with the amino acid sequence shown in SEQ ID NO: 1, wherein the improved characteristics include an increased pH stability and/or an increased thermostability.

In some embodiments of the present disclosure, the pH stability includes significant acid resistance after being stored at pH 4.0 for 2 h.

In some embodiments of the present disclosure, the increased thermostability includes increased stability at 70-99° C., and/or enhanced stability after being diluted 10-fold with a buffer at pH 4.5 and subjected to heat treatment at 95° C.

In some embodiments of the present disclosure, the Chi3 angle of the disulfide bond site pair is 65°<Chi3<120° or the Chi3 angle is −65°>Chi3>−120°.

In some embodiments of the present disclosure, compared with the parental α-amylase with the amino acid sequence shown in SEQ ID NO: 1, the α-amylase mutant has the disulfide bond site pair mutation as shown in any one of (1) to (43):
(1) Q97C, D227C; (2) P346C, K381C; (3) A2C, E416C; (4) E120C, S131C; (5) N127C, G192C; (6) D184C, K238C; (7) P243C, T281C; (8) Y266C, N291C; (9) V325C, A348C; (10) W183C, D195C; (11) G66C, T76C; (12) Y99C, G228C; (13) Q356C, Q397C; (14) T140C, A200C; (15) M204C, F241C; (16) V221C, T253C; (17) D18C, S53C; (18) Y60C, G109C; (19) Y394C, V414C; (20) W183C, N193C; (21) S297C, D341C; (22) D286C, L313C; (23) G301C, D428C; (24) A110C, W139C; (25) E412C, G439C; (26) V116C, A138C; (27) A27C, A90C; (28) G108C, Y199C; (29) S433C, K469C; (30) E120C, R174C; (31) P384C, T451C; (32) T410C, M436C; (33) G20C, G79C; (34) G408C, L425C; (35) F12C, P44C; (36) L22C, Y78C; (37) L289C, T312C; (38) T410C, A423C; (39) D286C, T323C; (40) L450C, V479C; (41) V116C, W158C; (42) M314C, E357C; (43) Q10C, W42C.

The present disclosure also provides a nucleic acid molecule.

Specifically, a nucleic acid molecule, wherein the nucleic acid molecule encodes the aforementioned α-amylase mutant.

The present disclosure also provides a recombinant expression vector.

Specifically, the present disclosure also provides a recombinant expression vector, wherein the recombinant expression vector comprises the aforementioned nucleic acid molecule.

In some embodiments of the present disclosure, a vector of the recombinant expression vector is a plasmid; preferably, the plasmid includes a pBE-S plasmid.

The present disclosure also provides a recombinant bacterium.

Specifically, the present disclosure also provides a recombinant bacterium, wherein the recombinant bacterium comprises the aforementioned nucleic acid molecule or the recombinant expression vector.

In some embodiments of the present disclosure, the recombinant bacterium is selected from Escherichia coli cell or Bacillus cell.

The present disclosure also provides an enzyme-containing composition.

Specifically, the present disclosure also provides an enzyme-containing composition, wherein the enzyme-containing composition comprises the aforementioned α-amylase mutant.

The present disclosure also provides a use of the aforementioned α-amylase mutant.

Specifically, the present disclosure also provides a use of the aforementioned α-amylase mutant in the production of syrup and/or fermentation products, such as sugar making, brewing, or other deep processing using starch as the raw material.

In some embodiments of the present disclosure, the process of producing syrup and/or fermentation products includes the following steps: (a) liquefying starch-containing materials in the presence of the α-amylase mutant mentioned above; (b) saccharifying the liquefied material; and (c) fermenting with fermenting organisms.

Compared with the prior art, the beneficial effects of the present disclosure are:
compared with the parent α-amylase, the α-amylase mutant provided in the present disclosure has significantly improved heat resistance and/or acid resistance. Most of the α-amylase mutants simultaneously possess excellent acid resistance and heat resistance, as well as higher catalytic activity for starch hydrolysis, which can better meet the needs of starch-based deep processing industries such as sugar making, brewing, alcohol production, and organic acid production.

### BRIEF DESCRIPTION OF THE DRAWINGS

FIG. 1 is a schematic diagram of Chi3 angle in an embodiment of the present disclosure.

FIG. 2 is a sequence homology comparison between the parental α-amylase (SEQ_01, amino acid sequence shown as SEQ ID NO.1) and PDB ID: 4UZU (4UZU_A, amino acid sequence shown as SEQ ID NO.2).

FIG. 3 is a test result diagram of the heat resistance of an α-amylase mutant.

FIG. 4 is a test result diagram of the acid resistance of an α-amylase mutant.

FIG. 5 is a test result diagram of the thermostability of an α-amylase mutant under acidic conditions.

FIG. 6 is an experimental result diagram of the liquefaction of corn starch.

FIG. 7 is an experimental result diagram of the liquefaction of cornmeal for ethanol production.

### DETAILED DESCRIPTION OF THE EMBODIMENTS

In order to enable a person skilled in the art to clearly understand the technical scheme of the present disclosure, the following embodiments are listed for description. It should be noted that the following embodiments do not limit the scope of protection of the present disclosure.

Unless otherwise specified, all biological materials, reagents, or devices used in the following embodiments can be obtained from conventional commercial sources or through existing known methods. Molecular biology experimental methods that are not specifically described in the following embodiments are all referenced to the specific methods listed in the “Molecular Cloning: A Laboratory Manual” (Third Edition) by Joseph Sambrook, or are carried out according to the instructions in the reagent kits and product manuals.

### Definition

α-Amylase: The term “α-amylase” refers to 1,4-α-D-glucan glucanohydrolase (EC.3.2.1.1), which catalyzes the hydrolysis of starch as well as other linear and branched 1,4-glucoside oligosaccharides and polysaccharides. The α-amylase activity can be determined using methods known in the art, such as the iodine test method described in “Alpha-amylase Preparation GB/T 24401-2009”.

According to the present disclosure, a variant that exhibits improved characteristics under at least one tested condition is considered to have improved characteristics compared to the parental α-amylase. For the purposes of the present disclosure, in some embodiments of the present disclosure, the improved characteristics are increased pH stability, for example, increased stability at acidic ambient temperatures, in order to meet the requirements of deep processing industries such as sugar making, brewing, alcohol production, and organic acid production that utilize starch as their raw material. In some embodiments of the present disclosure, the improved characteristics are increased thermostability, for example, increased stability at high temperatures. In some embodiments of the present disclosure, the improved characteristics are both increased thermostability and increased pH stability, for instance, both increased stability at acidic ambient temperatures and increased stability at high temperatures.

Compared to the parental α-amylase, some α-amylase mutants of the present disclosure exhibit increased stability at temperatures ranging from 70 to 99° C., as well as significant acid resistance after being stored at pH 4.0 for 2 h. Additionally, these mutants also exhibit enhanced stability after being diluted 10-fold with a buffer at pH 4.5 and subjected to heat treatment at 95° C. Compared to the parental α-amylase, some α-amylase mutants of the present disclosure exhibit increased stability at temperatures ranging from 70 to 99° C. Compared to the parental α-amylase, some α-amylase mutants of the present disclosure exhibit significant acid resistance after being stored at pH 4.0 for 2 h. Compared to the parental α-amylase, some α-amylase mutants of the present disclosure also exhibit enhanced stability after being diluted 10-fold with a buffer at pH 4.5 and subjected to heat treatment at 95° C. It is understood that, compared to the parental α-amylase, the α-amylase mutants of the present disclosure have at least one advantage in terms of either heat resistance or acid resistance.

Parent or parental α-amylase: The term “parent” or “parental α-amylase” refers to an α-amylase with the amino acid sequence as shown in SEQ ID NO:1.

Variant, mutant: The terms “variant” and “mutant” refer to polypeptides with α-amylase activity that contain mutations (i.e., substitutions, insertions, and/or deletions) at one or more (e.g., several) positions relative to the parental α-amylase as shown in SEQ ID NO:1. Substitution refers to the replacement of an amino acid occupying a specific position with a different amino acid; deletion refers to the removal of the amino acid occupying a specific position; and insertion refers to the addition of an amino acid immediately adjacent to and following the amino acid occupying a specific position. The mutants of the present disclosure have at least 20% of the α-amylase activity of the mature polypeptide in SEQ ID NO:1, for instance, at least 30%, at least 40%, at least 50%, at least 60%, at least 70%, at least 80%, at least 90%, at least 95%, at least 96%, at least 97%, at least 98%, at least 99%, or at least 100% of the α-amylase activity.

Encoding sequence: The term “encoding sequence” or “coding region” refers to a polynucleotide sequence that specifies the amino acid sequence of a polypeptide. The boundaries of the encoding sequence are generally determined by the open reading frame (ORF), which typically begins with an ATG start codon or alternative start codons (such as GTG and TTG) and ends with a stop codon (such as TAA, TAG, and TGA). Encoding sequences can be sequences of genomic DNA, cDNA, synthetic polynucleotides, and/or recombinant polynucleotides.

Control sequence: The term “control sequence” refers to nucleic acid sequences necessary for the expression of a polypeptide. Control sequences can be either natural or exogenous to the polynucleotide encoding the polypeptide, and they can be either natural or exogenous to each other. Such control sequences include, but are not limited to, leader sequences, polyadenylation sequences, propeptide sequences, promoter sequences, signal peptide sequences, and transcription terminator sequences. These control sequences may provide multiple linkers for the purpose of introducing specific restriction sites that facilitate the connection of the control sequences to the coding region of the polynucleotide encoding the polypeptide.

Expression: The term “expression” includes any step involved in the production of a polypeptide, including but not limited to transcription, post-transcriptional modification, translation, post-translational modification, and secretion. Expression can be measured, for example, to detect increased expression, by techniques known in the art, measuring the levels of mRNA and/or translated polypeptide.

Expression vector: The term “expression vector” refers to a linear or circular DNA molecule that contains a polynucleotide encoding a polypeptide and is operably linked to control sequences that provide for its expression.

Fermentable medium: The term “fermentable medium” or “fermentation medium” refers to a medium containing one or more (e.g., two, several) sugars such as glucose, fructose, sucrose, cellobiose, xylose, xylulose, arabinose, mannose, galactose, and/or soluble oligosaccharides, wherein the medium is capable of being partially converted (fermented) by host cells into desired products, such as ethanol. In some cases, the fermentation medium is derived from natural sources such as sugarcane, starch, or cellulose; and it can be pretreated by enzymatic hydrolysis (saccharification) of these sources. The term fermentation medium is understood herein to refer to the medium prior to the addition of fermenting organisms, for example, the medium resulting from a saccharification process, as well as the medium used in simultaneous saccharification and fermentation (SSF) processes.

For the purpose of this description, the Needleman-Wunsch algorithm (Needleman and Wunsch, J. Mol. Biol. [Journal of Molecular Biology] 1970, 48, 443-453) is used to determine the degree of sequence identity between two amino acid sequences, as implemented in the Needle program of the EMBOSS software package (EMBOSS: The European Molecular Biology Open Software Suite, Rice et al., Trends Genet. [Trends in Genetics] 2000, 16, 276-277) (preferably version 3.0.0 or later). The optional parameters used are a gap opening penalty (GOP) of 10, a gap extension penalty (GEP) of 0.5, and the EBLOSUM62 (EMBOSS version of BLOSUM62) substitution matrix (SM). The “longest identity” output from Needle (obtained using the -nobrief option) is used as the percentage identity and calculated as follows:

(Number of Identical Residues×100)/(Length of the Reference Sequence−Total Number of Gaps in the Alignment)

Variant naming convention: For the purpose of the present disclosure, the polypeptide disclosed in SEQ ID NO: 1 is used to identify corresponding amino acid residues in other α-amylases. The amino acid sequence of the α-amylase mutant is aligned with the polypeptide disclosed in SEQ ID NO: 1 using the Needleman-Wunsch algorithm (1970, J. Mol. Biol. 48:443-453) implemented in the Needle program of the EMBOSS package (EMBOSS: The European Molecular Biology Open Software Suite, Rice et al., 2000, Trends Genet. 16:276-277) (preferably version 5.0.0 or later). This alignment determines the amino acid position numbering corresponding to any amino acid residue in the mature polypeptide disclosed in SEQ ID NO: 1.

When describing mutants, for ease of reference, the accepted IUPAC one-letter or three-letter amino acid abbreviations are adopted.

Substitution: For amino acid substitutions, the following nomenclature is used: original amino acid, position, substituted amino acid. For example, the substitution of glutamine by cysteine at position 97 is denoted as “Gln97Cys” or “Q97C”.

Amino acid site number difference value: According to the present disclosure, “amino acid site number difference value “refers to the difference between the site numbers of two amino acids, which represents the distance between the two amino acids in the sequence, i.e., the number of amino acids that exist between them. In the embodiments of the present disclosure, when it is stated that the amino acid site number difference value for a disulfide bond site pair is greater than 10, it means that there are more than 10 amino acids separating the two amino acids forming the disulfide bond. For example, in the disulfide bond pair Q97C and D227C, there are 130 amino acids separating the two amino acids forming the disulfide bond.

Cysteine SG atom: refers to the gamma sulfur atom of cysteine. The sulfhydryl groups of two cysteine residues on the peptide chain undergo an oxidation reaction, causing the formation of a covalent bond between the gamma sulfur atoms, that is, a disulfide bond. In some embodiments of the present disclosure, the distance between the two cysteine SG atoms forming the disulfide bond is within 5 Å range; in some embodiments of the present disclosure, the distance between the two cysteine SG atoms forming the disulfide bond is within 4.5 Å range; in some embodiments of the present disclosure, the distance between the two cysteine SG atoms forming the disulfide bond is within 4 Å; in some embodiments of the present disclosure, the distance between the two cysteine SG atoms forming the disulfide bond is within 3.5 Å range; in some embodiments of the present disclosure, the distance between the two cysteine SG atoms forming the disulfide bond is within 3 Å; and in some embodiments of the present disclosure, the distance between the two cysteine SG atoms forming the disulfide bond is within 2.5 Å range.

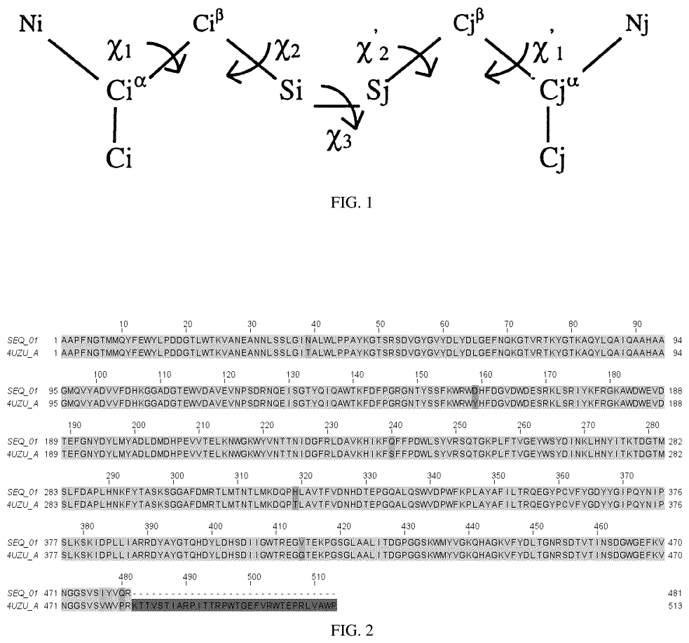

Chi3 angle: Abbreviated as “_{χ}3”, where “_{χ}” is the 22nd letter in the Greek alphabet. The Chi3 angle refers to the dihedral angle defined by the torsion of Ciβ-Si-Sj-Cjβ, as shown in FIG. 1. In embodiments of the present disclosure, the Chi3 angle of the disulfide bond site pair is 60°<Chi3<120° or −60°>Chi3>−120°. In some embodiments of the present disclosure, the Chi3 angle of the disulfide bond site pair is 65°<Chi3<120° or the Chi3 angle is −65°<Chi3<−120°.

### Embodiment 1: Rational Design of Amylase

### 1.1 Ab Initio Modelling

The parental α-amylase gene sequence (SEQ ID NO: 1) of the present disclosure exhibits the lowest proportion of essential cysteines necessary for disulfide bond pair formation, with only one present, indicating significant potential for disulfide bond design. Using the BLAST server within the GenBank database (http://www.ncbi.nlm.nih.gov/genbank/), sequence searches and homology alignments revealed that Alpha-amylase (PDB ID: 4UZU), also derived from Geobacillus stearothermophilus, shares 98.34% homology with the amylase sequence of the present disclosure. The parental α-amylase (SEQ ID NO: 1) of the present disclosure belongs to the CAZY family GH13, which is the most extensively studied among all glycoside hydrolase families. Mechanistically, α-amylase hydrolyzes the α-1,4-glycosidic bonds in starch through a covalent glycosyl-enzyme intermediate. This mechanism is well-established and requires a catalytic nucleophile, a Brønsted acid/base, and an “auxiliary” residue for completion. As shown in FIG. 2, the parental α-amylase (SEQ ID NO: 1) of the present disclosure shares 98.34% sequence homology with PDB ID: 4UZU, confirming that the key catalytic active sites are similarly composed of the catalytic triad Asp232, Glu262, and Asp329. A primary feature of the disulfide bond design in the present disclosure is that the disulfide bond site pairs are not located within 5 Å range of the active site of the α-amylase mutant, thereby avoiding any impact on the substrate catalytic activity of α-amylase.

AlphaFold2 is a deep neural network learning model based on the Transformer architecture, providing end-to-end protein structure prediction with extremely high modeling accuracy. Leveraging this platform, the AlphaFold2 computing service is established to input protein sequences for predicting the 3D structure of enzymes. After the completion of the operation, five structural files are generated, among which ranked_0 is the highest-scoring structural model with a pLDDT value of 98.55 and an RMSD difference of 0.715 compared with the resolved structure 4UZU. Therefore, the most credible structural model is selected for further analysis.

### 1.2 Assessment of Structural Conformation Rationality

The structural conformation rationality of the structural model obtained from the previous steps was evaluated using the SAVES v6.0 (https://saves.mbi.ucla.edu/) scoring program, and the results were presented in the form of a Ramachandran plot. Generally, if the proportion of amino acid residues falling within the allowed and maximally allowed regions exceeds 90% of the entire protein, the conformation of the model can be considered to conform to the rules of stereochemistry. A model with over 90% of its residues in the most favorable region is considered high-quality, while a reliable model should have more than 80% of its residues in these regions. The evaluation results are as follows: The overall structural quality score of the model predicted by AlphaFold2 is 95.7717, and the Ramachandran plot shows that 87.0% of the residues in the predicted model are located in the most favorable region. The results indicate that the model predicted by AlphaFold2 possesses extremely high accuracy and comprehensively intuitive rational conformation. Therefore, the model predicted by AlphaFold2 can be selected for subsequent experiments.

### 1.3 Disulfide Bond Design

To identify rational and effective disulfide bond pairs for enhancing the thermostability of amylase, this approach employs a deep learning algorithm model, coupled with predetermined criteria to screen for disulfide bond pairs: a) the disulfide bond site pairs are not within 5 A of the active region; b) the amino acid sequence difference value between the disulfide bond site pairs is greater than 10; c) after mutation, the distance between cysteine SG atoms after the disulfide bond site pair mutation is within 5 A; d) after the disulfide bond site pair mutation, the Chi3 angle is between 80<Chi3<120 or −80>Chi3>−120. The final compilation of computational results is shown in Table 1. Based on these comprehensive conditions, a total of 43 pairs of candidate disulfide bond mutation libraries have been screened, which can be experimentally verified;

TABLE 1

| | | | | | Within |
|---|---|---|---|---|---|
| | | | Distance | | 5A of |
| Amino acid | Disulfide bond site | Sequence | Å after | CHi3 | active |
| sequence | pair mutation | distance | mutation | angle | site |
| Parental | / | / | / | / | / |
| α-amylase | | | | | |
| α-amylase | (1) Q97C, D227C | 130 | 4.7 | 82.07 | No |
| mutant 1 | | | | | |
| α-amylase | (2) P346C, K381C | 35 | 2.05 | 97.81 | No |
| mutant 2 | | | | | |
| α-amylase | (3) A2C, E416C | 414 | 4.85 | 89.97 | No |
| mutant 3 | | | | | |
| α-amylase | (4) E120C, S131C | 11 | 3.55 | 97.19 | No |
| mutant 4 | | | | | |
| α-amylase | (5) N127C, G192C | 65 | 2.08 | 79.7 | No |
| mutant 5 | | | | | |
| α-amylase | (6) D184C, K238C | 54 | 4.1 | 74.43 | No |
| mutant 6 | | | | | |
| α-amylase | (7) P243C, T281C | 38 | 2.28 | 97.19 | No |
| mutant 7 | | | | | |
| α-amylase | (8) Y266C, N291C | 25 | 2.16 | 118.21 | No |
| mutant 8 | | | | | |
| α-amylase | (9) V325C, A348C | 23 | 2.05 | 68.04 | No |
| mutant 9 | | | | | |
| α-amylase | (10) W183C, D195C | 12 | 4.78 | 92.93 | No |
| mutant 10 | | | | | |
| α-amylase | (11) G66C, T76C | 10 | 2.14 | 99.74 | No |
| mutant 11 | | | | | |
| α-amylase | (12) Y99C, G228C | 129 | 2.15 | 118.37 | No |
| mutant 12 | | | | | |
| α-amylase | (13) Q356C, Q397C | 41 | 4.22 | 112.39 | No |
| mutant 13 | | | | | |
| α-amylase | (14) T140C, A200C | 60 | 3.37 | 83.91 | No |
| mutant 14 | | | | | |
| α-amylase | (15) M204C, F241C | 37 | 3.16 | 100.77 | No |
| mutant 15 | | | | | |
| α-amylase | (16) V221C, T253C | 32 | 3.15 | 84.76 | No |
| mutant 16 | | | | | |
| α-amylase | (17) D18C, S53C | 35 | 3.35 | 70.38 | No |
| mutant 17 | | | | | |
| α-amylase | (18) Y60C, G109C | 49 | 3.49 | 117.96 | No |
| mutant 18 | | | | | |
| α-amylase | (19) Y394C, V414C | 20 | 3.13 | 87.78 | No |
| mutant 19 | | | | | |
| α-amylase | (20) W183C, N193C | 10 | 3.89 | 100.86 | No |
| mutant 20 | | | | | |
| α-amylase | (21) S297C, D341C | 44 | 3.56 | −89.74 | No |
| mutant 21 | | | | | |
| α-amylase | (22) D286C, L313C | 27 | 4.94 | −83.26 | No |
| mutant 22 | | | | | |
| α-amylase | (23) G301C, D428C | 127 | 4.87 | −110.58 | No |
| mutant 23 | | | | | |
| α-amylase | (24) A110C, W139C | 29 | 5 | −76.77 | No |
| mutant 24 | | | | | |
| α-amylase | (25) E412C, G439C | 27 | 3.61 | −74.56 | No |
| mutant 25 | | | | | |
| α-amylase | (26) V116C, A138C | 22 | 4.66 | −68.86 | No |
| mutant 26 | | | | | |
| α-amylase | (27) A27C, A90C | 63 | 3.66 | −65.01 | No |
| mutant 27 | | | | | |
| α-amylase | (28) G108C, Y199C | 91 | 2.18 | 113.91 | No |
| mutant 28 | | | | | |
| α-amylase | (29) S433C, K469C | 36 | 2.37 | −113.53 | No |
| mutant 29 | | | | | |
| α-amylase | (30) E120C, R174C | 54 | 4.4 | −101.89 | No |
| mutant 30 | | | | | |
| α-amylase | (31) P384C, T451C | 67 | 3.42 | −97.98 | No |
| mutant 31 | | | | | |
| α-amylase | (32) T410C, M436C | 26 | 4.43 | −86.59 | No |
| mutant 32 | | | | | |
| α-amylase | (33) G20C, G79C | 59 | 1.84 | −102.66 | No |
| mutant 33 | | | | | |
| α-amylase | (34) G408C, L425C | 17 | 3.9 | −118.39 | No |
| mutant 34 | | | | | |
| α-amylase | (35) F12C, P44C | 32 | 3.89 | −98.87 | No |
| mutant 35 | | | | | |
| α-amylase | (36) L22C, Y78C | 56 | 4.07 | −109.9 | No |
| mutant 36 | | | | | |
| α-amylase | (37) L289C, T312C | 23 | 3.91 | −86.17 | No |
| mutant 37 | | | | | |
| α-amylase | (38) T410C, A423C | 13 | 2.76 | −96.22 | No |
| mutant 38 | | | | | |
| α-amylase | (39) D286C, T323C | 37 | 4.56 | −107.95 | No |
| mutant 39 | | | | | |
| α-amylase | (40) L450C, V479C | 29 | 4.36 | −63.84 | No |
| mutant 40 | | | | | |
| α-amylase | (41) V116C, W158C | 42 | 4.61 | −80.79 | No |
| mutant 41 | | | | | |
| α-amylase | (42) M314C, E357C | 43 | 4.75 | −87.32 | No |
| mutant 42 | | | | | |
| α-amylase | (43) Q10C, W42C | 32 | 4.89 | −119.54 | No |
| mutant 43 | | | | | |

### Embodiment 2. Construction and Expression of Amylase and its Variants

### 2.1 Materials and Reagents

### 2.1.1 Strains and Vectors

Expression strains containing the amylase gene amyS and its variants, Escherichia coli TOP10, Bacillus WB600, vector pBE-S, antibiotics, kanamycin, and ampicillin were all purchased from Sangon Biotech (Shanghai) Co., Ltd.

### 2.1.2 Enzymes and Kits

Q5® High-Fidelity 2× Master Mix PCR polymerase, restriction enzymes, and other enzymes were purchased from New England Biolabs (NEB). Plasmid extraction kits and purification kits were purchased from Sangon Biotech (Shanghai) Co., Ltd.

### 2.1.3 Medium

The medium for Escherichia coli is LB medium (1% tryptone, 0.5% yeast extract, 1% NaCl, pH 7.0). LB+Amp medium is LB medium supplemented with ampicillin at a final concentration of 100 μg/mL. The medium for Bacillus is TB medium (11.8 g/L tryptone, 23.6 g/L yeast extract, 9.4 g/L K₂HPO₄, 2.2 g/L KH₂PO₄). TB+Kan medium is TB medium supplemented with kanamycin at a final concentration of 20 μg/mL.

### 2.1.4 Chemical Reagents

Soluble starch was purchased from Huzhou Zhaowang Pharmaceutical Co., Ltd. Other reagents were purchased from Guangzhou Chemical Reagent Factory.

### 2.2 Determination Method of Amylase Activity

The amylase activity is determined by the iodine test method specified in the national standard “Alpha-amylase Preparation GB/T 24401-2009.” The Alpha-amylase preparation can randomly cleave the α-1,4-glucosidic bonds in the starch molecular chain into short-chain dextrins of varying lengths, as well as a small amount of maltose and glucose, causing the characteristic blue-violet reaction of starch to iodine to gradually disappear and turning into a brownish red color. The rate of color disappearance is related to the enzyme activity, and based on this, the enzyme activity can be calculated through the absorbance after the reaction.

One enzyme activity unit (U/g or U/mL) is defined as the amount of solid enzyme powder (1 g) or liquid enzyme (1 mL) that can liquefy 1 mg of soluble starch at 70° C. and a pH of 6.0 within 1 min.

In cases involving genetic mutations, the parent sequence and its species should be disclosed, and subsequent site numbering is based on this parent sequence. The mutated α-amylase (amyS) gene (Genebank, AGK25234.1) of Geobacillus stearothermophilus, its amino acid sequence is shown in SEQ ID NO.1.

### 2.2 Expression of Amylase and its Variants

### 2.2.1 Synthesis of High-Temperature Amylase Amys Gene and Vector Construction

The mutated high-temperature α-amylase (amyS) gene (Genebank, AGK25234.1) of Geobacillus stearothermophilus, with its corresponding amino acid sequence of the α-amylase as shown in SEQ ID NO.1 (designated as the parental α-amylase).

At the 5′ and 3′ ends of the high-temperature α-amylase amyS gene, restriction enzyme sites for Ndel and Sall were introduced, respectively. This modified gene was then ligated into the pUC57-amp vector. The pUC57-amyS construct was inoculated into LBA medium and cultured overnight. Following cultivation, the plasmid was extracted and subjected to restriction enzyme digestion using Ndel and SalI. The target gene fragment was recovered from the agarose gel and subsequently ligated into the expression vector pBE-S, resulting in the expression vector pBE-amyS.

### 2.2.2 Introduction of Mutations in Disulfide Bonds

Using the pBE-amyS as a template, the first cysteine was introduced into the amylase amyS gene via PCR. Following DpnI (DpnI endonuclease) digestion, the target fragment was recovered from the agarose gel. The digested product was then transformed into Escherichia coli TOP10 competent cells using a chemical transformation heat shock method. The recombinant transformants were verified through colony PCR, and the plasmids from verified transformants were extracted and sequenced to confirm the corresponding mutant, resulting in the expression vector pBE-amyS-M1-1. Subsequently, using pBE-amyS-M1-1 as a template, the second cysteine was introduced into the amylase amyS gene via PCR. Following DpnI (DpnI endonuclease) digestion, the target fragment was recovered from the agarose gel. The digested product was then transformed into Escherichia coli TOP10 competent cells using a chemical transformation heat shock method. The recombinant transformants were verified through colony PCR, and the plasmids from verified transformants were extracted and sequenced to confirm the corresponding mutant, resulting in the expression vector pBE-amyS-M1 containing a pair of cysteine. Finally, the pBE-amyS-M1 vector was transformed into WB600 chemically competent cells using the chemical transformation method, resulting in the recombinant Bacillus transformants.

### 2.2.3 Flask Fermentation of Mutant Strains Containing Disulfide Bonds

Using toothpicks, individually pick the recombinant transformants obtained from Step 2 and transfer them into 250 mL flasks, each containing 40 mL of TB medium. Cultivate the flasks at 37° C., 220 rpm, with 85% humidity for approximately 48 h. Centrifuge to obtain the supernatant, and then purify both the parental α-amylase gene and the mutants separately using affinity chromatography purification, resulting in enzyme solutions of each α-amylase mutant.

### Product Effect Testing

### 1. Detection of Optimal Reaction pH for α-Amylase Mutants

Under the condition of 70° C., the enzyme activities of amylase were measured at pH values of 4.0, 4.5, 6.0, and 7.0, respectively. The results are shown in Table 2.

Among them, the enzyme activity of the parental α-amylase measured at pH 6.0 was used as the control to calculate the relative enzyme activities of each mutant enzyme under different pH conditions.

Relative enzyme activity refers to the ratio of the enzyme activity under certain conditions to the enzyme activity under optimal reaction conditions. The formula for calculating relative enzyme activity is as follows: Relative Enzyme Activity=(Enzyme Activity Measured/Enzyme Activity at Optimal Reaction Conditions)×100%.

TABLE 2

| | pH | | | | |
|---|---|---|---|---|---|
| Group | pH 4 | pH 4.5 | pH 5 | pH 6 | pH 7 |
| Parental α-amylase | 47% | 71% | 96% | 100% | 66% |
| α-amylase mutant 1 | 62% | 94% | 100% | 100% | 66% |
| α-amylase mutant 2 | 50% | 70% | 98% | 100% | 63% |
| α-amylase mutant 3 | 57% | 74% | 92% | 100% | 65% |
| α-amylase mutant 4 | 49% | 85% | 98% | 100% | 66% |
| α-amylase mutant 5 | 55% | 76% | 97% | 100% | 63% |
| α-amylase mutant 6 | 61% | 88% | 93% | 100% | 67% |
| α-amylase mutant 7 | 58% | 77% | 95% | 100% | 60% |
| α-amylase mutant 8 | 55% | 73% | 97% | 100% | 62% |
| α-amylase mutant 9 | 44% | 69% | 94% | 100% | 70% |
| α-amylase mutant 10 | 57% | 83% | 99% | 100% | 61% |
| α-amylase mutant 11 | 56% | 85% | 100% | 100% | 63% |
| α-amylase mutant 12 | 46% | 68% | 95% | 100% | 64% |
| α-amylase mutant 13 | 53% | 74% | 92% | 100% | 70% |
| α-amylase mutant 14 | 53% | 77% | 100% | 100% | 61% |
| α-amylase mutant 15 | 52% | 84% | 100% | 100% | 60% |
| α-amylase mutant 16 | 53% | 86% | 100% | 100% | 70% |
| α-amylase mutant 17 | 60% | 72% | 93% | 100% | 67% |
| α-amylase mutant 18 | 45% | 78% | 97% | 100% | 64% |
| α-amylase mutant 19 | 60% | 77% | 100% | 100% | 67% |
| α-amylase mutant 20 | 46% | 73% | 98% | 100% | 68% |
| α-amylase mutant 21 | 51% | 71% | 96% | 100% | 65% |
| α-amylase mutant 22 | 48% | 77% | 93% | 100% | 61% |
| α-amylase mutant 23 | 57% | 78% | 94% | 100% | 62% |
| α-amylase mutant 24 | 46% | 74% | 97% | 100% | 68% |
| α-amylase mutant 25 | 57% | 70% | 93% | 100% | 69% |
| α-amylase mutant 26 | 50% | 78% | 97% | 100% | 65% |
| α-amylase mutant 27 | 58% | 84% | 96% | 100% | 64% |
| α-amylase mutant 28 | 62% | 82% | 93% | 100% | 70% |
| α-amylase mutant 29 | 49% | 79% | 100% | 100% | 67% |
| α-amylase mutant 30 | 60% | 94% | 98% | 100% | 60% |
| α-amylase mutant 31 | 61% | 83% | 94% | 100% | 63% |
| α-amylase mutant 32 | 60% | 75% | 95% | 100% | 67% |
| α-amylase mutant 33 | 62% | 79% | 95% | 100% | 62% |
| α-amylase mutant 34 | 57% | 82% | 92% | 100% | 61% |
| α-amylase mutant 35 | 51% | 71% | 95% | 100% | 65% |
| α-amylase mutant 36 | 48% | 69% | 98% | 100% | 62% |
| α-amylase mutant 37 | 52% | 75% | 95% | 100% | 61% |
| α-amylase mutant 38 | 57% | 82% | 92% | 100% | 66% |
| α-amylase mutant 39 | 47% | 73% | 97% | 100% | 68% |
| α-amylase mutant 40 | 62% | 86% | 100% | 100% | 60% |
| α-amylase mutant 41 | 51% | 76% | 98% | 100% | 69% |
| α-amylase mutant 42 | 59% | 77% | 99% | 100% | 61% |
| α-amylase mutant 43 | 56% | 75% | 99% | 100% | 69% |

From Table 2, it can be seen that the optimal reaction pH for the α-amylase mutants is 6, and most of the α-amylase mutants also exhibit high enzyme activities at pH 4 and pH 5.

### 2. Detection of Optimal Reaction Temperature for α-Amylase Mutants

Under the condition of pH 6.0, the enzyme activities of both the parental α-amylase and the α-amylase mutants were measured at temperatures of 60° C., 70° C., 80° C., and 90° C., respectively. The results are shown in Table 3.

Among them, the enzyme activity of the parental α-amylase measured at 70° C. was used as the control to calculate the relative enzyme activities of each enzyme under different temperature conditions.

Relative enzyme activity refers to the ratio of the enzyme activity under certain conditions to the enzyme activity under optimal reaction conditions. The formula for calculating relative enzyme activity is as follows: Relative Enzyme Activity=(Enzyme Activity Measured/Enzyme Activity at Optimal Reaction Conditions)×100%.

TABLE 3

| | Temperature | | | |
|---|---|---|---|---|
| Group | 60° C. | 70° C. | 80° C. | 90° C. |
| Parental α-amylase | 60% | 100% | 80% | 67% |
| α-amylase mutant 1 | 65% | 100% | 94% | 90% |
| α-amylase mutant 2 | 60% | 100% | 80% | 81% |
| α-amylase mutant 3 | 64% | 100% | 78% | 75% |
| α-amylase mutant 4 | 61% | 100% | 99% | 75% |
| α-amylase mutant 5 | 62% | 100% | 78% | 76% |
| α-amylase mutant 6 | 60% | 100% | 90% | 82% |
| α-amylase mutant 7 | 66% | 100% | 96% | 71% |
| α-amylase mutant 8 | 64% | 100% | 90% | 70% |
| α-amylase mutant 9 | 63% | 100% | 90% | 84% |
| α-amylase mutant 10 | 66% | 100% | 75% | 63% |
| α-amylase mutant 11 | 62% | 100% | 75% | 65% |
| α-amylase mutant 12 | 60% | 100% | 99% | 83% |
| α-amylase mutant 13 | 61% | 100% | 81% | 66% |
| α-amylase mutant 14 | 61% | 100% | 81% | 67% |
| α-amylase mutant 15 | 58% | 100% | 82% | 70% |
| α-amylase mutant 16 | 67% | 100% | 84% | 71% |
| α-amylase mutant 17 | 59% | 100% | 91% | 69% |
| α-amylase mutant 18 | 58% | 100% | 92% | 75% |
| α-amylase mutant 19 | 62% | 100% | 76% | 72% |
| α-amylase mutant 20 | 65% | 100% | 83% | 77% |
| α-amylase mutant 21 | 66% | 100% | 90% | 74% |
| α-amylase mutant 22 | 59% | 100% | 91% | 77% |
| α-amylase mutant 23 | 58% | 100% | 90% | 65% |
| α-amylase mutant 24 | 63% | 100% | 76% | 63% |
| α-amylase mutant 25 | 67% | 100% | 87% | 67% |
| α-amylase mutant 26 | 59% | 100% | 79% | 83% |
| α-amylase mutant 27 | 62% | 100% | 100% | 89% |
| α-amylase mutant 28 | 60% | 100% | 88% | 74% |
| α-amylase mutant 29 | 58% | 100% | 90% | 80% |
| α-amylase mutant 30 | 67% | 100% | 91% | 74% |
| α-amylase mutant 31 | 63% | 100% | 85% | 68% |
| α-amylase mutant 32 | 59% | 100% | 93% | 68% |
| α-amylase mutant 33 | 67% | 100% | 94% | 86% |
| α-amylase mutant 34 | 63% | 100% | 100% | 90% |
| α-amylase mutant 35 | 65% | 100% | 94% | 81% |
| α-amylase mutant 36 | 65% | 100% | 78% | 70% |
| α-amylase mutant 37 | 59% | 100% | 80% | 67% |
| α-amylase mutant 38 | 62% | 100% | 78% | 81% |
| α-amylase mutant 39 | 65% | 100% | 77% | 62% |
| α-amylase mutant 40 | 67% | 100% | 77% | 60% |
| α-amylase mutant 41 | 63% | 100% | 89% | 80% |
| α-amylase mutant 42 | 58% | 100% | 97% | 73% |
| α-amylase mutant 43 | 64% | 100% | 90% | 69% |

From Table 3, it can be seen that the optimal reaction temperature for the α-amylase mutants is 70° C., and they also exhibit relatively high enzyme activities at 80° C. and 90° C.

### 3. Heat Resistance of α-Amylase Mutants

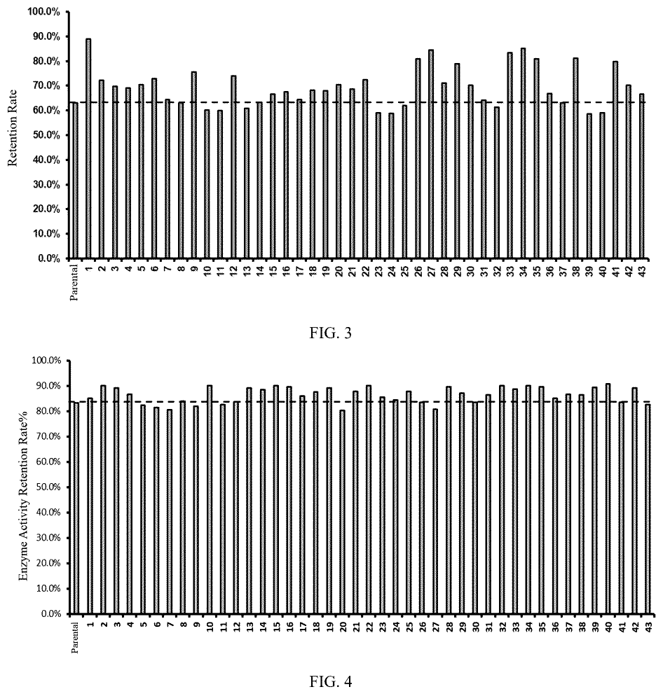

Purified enzyme solutions of both the parental α-amylase and the α-amylase mutants were subjected to heat treatment at 99° C. for 30 min, followed by cooling on ice. Subsequently, their enzyme activities were measured under the conditions of 70° C. and pH 6.0. The enzyme activity retention rate was calculated using the formula: Enzyme Activity Retention Rate=(Enzyme Activity After Heat Storage/Enzyme Activity Before Heat Storage)×100%. The results of the heat resistance test are shown in Table 4 and FIG. 3. In FIG. 3, the parental α-amylase is labeled as “Parental,” and the α-amylase mutants 1 to 43 are abbreviated as 1 to 43, respectively (the same applies below).

TABLE 4

| | Enzyme activity |
|---|---|
| | retention rate after |
| | treatment at 99° C. for |
| Group | 30 min |
| Parental α-amylase | 63.1% |
| α-amylase mutant 1 | 89.0% |
| α-amylase mutant 2 | 72.2% |
| α-amylase mutant 3 | 69.7% |
| α-amylase mutant 4 | 69.1% |
| α-amylase mutant 5 | 70.5% |
| α-amylase mutant 6 | 72.9% |
| α-amylase mutant 7 | 64.4% |
| α-amylase mutant 8 | 63.1% |
| α-amylase mutant 9 | 75.5% |
| α-amylase mutant 10 | 60.2% |
| α-amylase mutant 11 | 59.9% |
| α-amylase mutant 12 | 74.0% |
| α-amylase mutant 13 | 60.9% |
| α-amylase mutant 14 | 63.3% |
| α-amylase mutant 15 | 66.6% |
| α-amylase mutant 16 | 67.6% |
| α-amylase mutant 17 | 64.5% |
| α-amylase mutant 18 | 68.3% |
| α-amylase mutant 19 | 68.0% |
| α-amylase mutant 20 | 70.4% |
| α-amylase mutant 21 | 68.7% |
| α-amylase mutant 22 | 72.5% |
| α-amylase mutant 23 | 59.1% |
| α-amylase mutant 24 | 58.8% |
| α-amylase mutant 25 | 62.0% |
| α-amylase mutant 26 | 80.8% |
| α-amylase mutant 27 | 84.4% |
| α-amylase mutant 28 | 71.1% |
| α-amylase mutant 29 | 78.9% |
| α-amylase mutant 30 | 70.2% |
| α-amylase mutant 31 | 64.1% |
| α-amylase mutant 32 | 61.3% |
| α-amylase mutant 33 | 83.3% |
| α-amylase mutant 34 | 85.1% |
| α-amylase mutant 35 | 80.9% |
| α-amylase mutant 36 | 66.8% |
| α-amylase mutant 37 | 63.0% |
| α-amylase mutant 38 | 81.2% |
| α-amylase mutant 39 | 58.7% |
| α-amylase mutant 40 | 59.0% |
| α-amylase mutant 41 | 79.8% |
| α-amylase mutant 42 | 70.2% |
| α-amylase mutant 43 | 66.7% |

From Table 4 and FIG. 3, it can be seen that the enzyme activity retention rates of α-amylase mutants 1-7, 9, 12, 14-22, 26-31, 33-36, 38, and 41-43, after treatment at 99° C. for 30 min, were significantly higher than that of the parental α-amylase, indicating a substantial enhancement in their heat resistance.

### 4. Acid Resistance of α-Amylase Mutants

Purified enzyme solutions of both the parental α-amylase and the α-amylase mutants were stored at pH 4.0 for 2 h, followed by enzyme activity measurements under the conditions of 70° C. and pH 6.0. The enzyme activity retention rate was calculated using the formula: Enzyme Activity Retention Rate=(Enzyme Activity After Heat Storage/Enzyme Activity Before Heat Storage)×100%. The results of the acid resistance test are shown in Table 5 and FIG. 4.

TABLE 5

| | Enzyme activity |
|---|---|
| | retention rate |
| | after storage at |
| Group | pH 4.0 for 2 h |
| Parental α-amylase | 83.3% |
| α-amylase mutant 1 | 85.1% |
| α-amylase mutant 2 | 89.9% |
| α-amylase mutant 3 | 89.0% |
| α-amylase mutant 4 | 86.6% |
| α-amylase mutant 5 | 82.2% |
| α-amylase mutant 6 | 81.3% |
| α-amylase mutant 7 | 80.4% |
| α-amylase mutant 8 | 83.8% |
| α-amylase mutant 9 | 81.9% |
| α-amylase mutant 10 | 90.1% |
| α-amylase mutant 11 | 82.5% |
| α-amylase mutant 12 | 83.7% |
| α-amylase mutant 13 | 89.0% |
| α-amylase mutant 14 | 88.4% |
| α-amylase mutant 15 | 90.0% |
| α-amylase mutant 16 | 89.5% |
| α-amylase mutant 17 | 85.9% |
| α-amylase mutant 18 | 87.6% |
| α-amylase mutant 19 | 89.1% |
| α-amylase mutant 20 | 80.2% |
| α-amylase mutant 21 | 87.7% |
| α-amylase mutant 22 | 89.9% |
| α-amylase mutant 23 | 85.4% |
| α-amylase mutant 24 | 84.3% |
| α-amylase mutant 25 | 87.7% |
| α-amylase mutant 26 | 83.4% |
| α-amylase mutant 27 | 80.6% |
| α-amylase mutant 28 | 89.6% |
| α-amylase mutant 29 | 87.0% |
| α-amylase mutant 30 | 83.4% |
| α-amylase mutant 31 | 86.3% |
| α-amylase mutant 32 | 90.0% |
| α-amylase mutant 33 | 88.7% |
| α-amylase mutant 34 | 89.9% |
| α-amylase mutant 35 | 89.5% |
| α-amylase mutant 36 | 85.1% |
| α-amylase mutant 37 | 86.6% |
| α-amylase mutant 38 | 86.3% |
| α-amylase mutant 39 | 89.4% |
| α-amylase mutant 40 | 90.7% |
| α-amylase mutant 41 | 83.4% |
| α-amylase mutant 42 | 89.0% |
| α-amylase mutant 43 | 82.5% |

From Table 5 and FIG. 4, it can be seen that the enzyme activity retention rates of α-amylase mutants 1-4, 8, 10, 12-19, 21-26, and 28-42, after storage at pH 4.0 for 2 h, were significantly higher than that of the parental α-amylase, indicating a substantial enhancement in their acid resistance.

### 5. Thermostability of α-Amylase Mutants Under Acidic Conditions

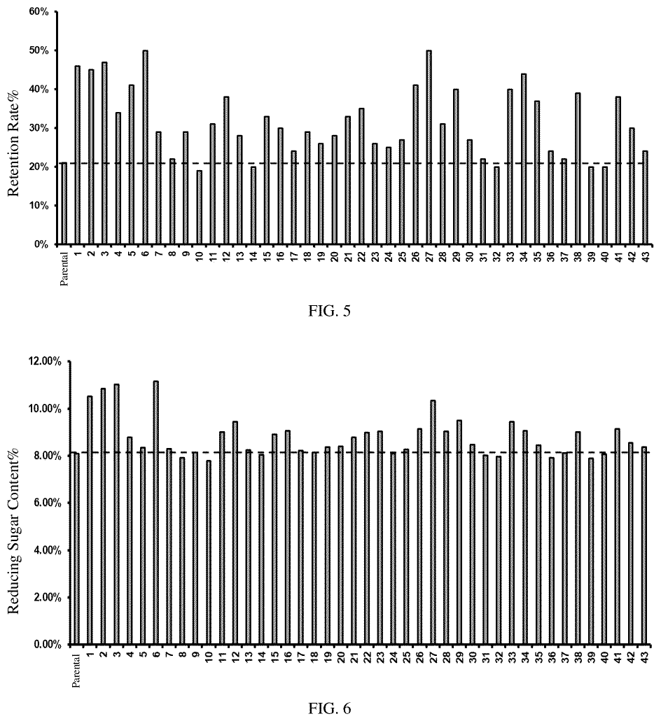

Purified enzyme solutions of both the parental α-amylase and the α-amylase mutants were diluted 10-fold with a pH 4.5 buffer. They were then subjected to heat treatment at 95° C. for 2 min, followed by cooling on ice. Subsequently, their enzyme activities were measured under the conditions of 70° C. and pH 6.0. The enzyme activity retention rate was calculated using the formula: Enzyme Activity Retention Rate=(Enzyme Activity After Heat Storage/Enzyme Activity Before Heat Storage)×100%. The results of the thermostability test are shown in Table 6 and FIG. 5.

TABLE 6

| | Enzyme activity |
|---|---|
| | retention rate after |
| | treatment at pH 4.5, |
| Group | 95° C. for 2 min |
| Parental α-amylase | 21% |
| α-amylase mutant 1 | 46% |
| α-amylase mutant 2 | 45% |
| α-amylase mutant 3 | 47% |
| α-amylase mutant 4 | 34% |
| α-amylase mutant 5 | 41% |
| α-amylase mutant 6 | 50% |
| α-amylase mutant 7 | 29% |
| α-amylase mutant 8 | 22% |
| α-amylase mutant 9 | 29% |
| α-amylase mutant 10 | 19% |
| α-amylase mutant 11 | 31% |
| α-amylase mutant 12 | 38% |
| α-amylase mutant 13 | 28% |
| α-amylase mutant 14 | 20% |
| α-amylase mutant 15 | 33% |
| α-amylase mutant 16 | 30% |
| α-amylase mutant 17 | 24% |
| α-amylase mutant 18 | 29% |
| α-amylase mutant 19 | 26% |
| α-amylase mutant 20 | 28% |
| α-amylase mutant 21 | 33% |
| α-amylase mutant 22 | 35% |
| α-amylase mutant 23 | 26% |
| α-amylase mutant 24 | 25% |
| α-amylase mutant 25 | 27% |
| α-amylase mutant 26 | 41% |
| α-amylase mutant 27 | 50% |
| α-amylase mutant 28 | 31% |
| α-amylase mutant 29 | 40% |
| α-amylase mutant 30 | 27% |
| α-amylase mutant 31 | 22% |
| α-amylase mutant 32 | 20% |
| α-amylase mutant 33 | 40% |
| α-amylase mutant 34 | 44% |
| α-amylase mutant 35 | 37% |
| α-amylase mutant 36 | 24% |
| α-amylase mutant 37 | 22% |
| α-amylase mutant 38 | 39% |
| α-amylase mutant 39 | 20% |
| α-amylase mutant 40 | 20% |
| α-amylase mutant 41 | 38% |
| α-amylase mutant 42 | 30% |
| α-amylase mutant 43 | 24% |

From Table 6 and FIG. 5, it can be seen that after being diluted 10-fold with a pH 4.5 buffer and subjected to heat treatment at 95° C. for 2 min, the enzyme activity retention rates of α-amylase mutants 1-9, 11-13, 15-31, 33-38, and 41-43 remained higher than that of the parental α-amylase, indicating a substantial enhancement in their thermostability under acidic conditions.

### 6. Liquefaction Experiment of Corn Starch

A 34% corn starch solution was prepared and its pH was adjusted to 4.5 using dilute sulfuric acid. A total of 500 g of the corn starch solution was added to the liquefaction tank, and 15 U/g of the parental α-amylase and each α-amylase mutant enzyme solution were added separately. The reaction process was set as follows: 50° C. for 1 min, followed by 70° C. for 5 min, and finally 95° C. for 70 min. After the reaction, the reducing sugar content of each experimental group was measured, and the results are shown in Table 7 and FIG. 6.

TABLE 7

| | Reducing sugar |
|---|---|
| Group | content |
| Parental α-amylase | 8.10% |
| α-amylase mutant 1 | 10.50% |
| α-amylase mutant 2 | 10.85% |
| α-amylase mutant 3 | 11.02% |
| α-amylase mutant 4 | 8.77% |
| α-amylase mutant 5 | 8.35% |
| α-amylase mutant 6 | 11.14% |
| α-amylase mutant 7 | 8.30% |
| α-amylase mutant 8 | 7.90% |
| α-amylase mutant 9 | 8.13% |
| α-amylase mutant 10 | 7.79% |
| α-amylase mutant 11 | 9.01% |
| α-amylase mutant 12 | 9.44% |
| α-amylase mutant 13 | 8.23% |
| α-amylase mutant 14 | 8.04% |
| α-amylase mutant 15 | 8.90% |
| α-amylase mutant 16 | 9.05% |
| α-amylase mutant 17 | 8.22% |
| α-amylase mutant 18 | 8.13% |
| α-amylase mutant 19 | 8.37% |
| α-amylase mutant 20 | 8.40% |
| α-amylase mutant 21 | 8.77% |
| α-amylase mutant 22 | 8.98% |
| α-amylase mutant 23 | 9.04% |
| α-amylase mutant 24 | 8.10% |
| α-amylase mutant 25 | 8.26% |
| α-amylase mutant 26 | 9.14% |
| α-amylase mutant 27 | 10.33% |
| α-amylase mutant 28 | 9.04% |
| α-amylase mutant 29 | 9.50% |
| α-amylase mutant 30 | 8.46% |
| α-amylase mutant 31 | 8.00% |
| α-amylase mutant 32 | 7.95% |
| α-amylase mutant 33 | 9.43% |
| α-amylase mutant 34 | 9.05% |
| α-amylase mutant 35 | 8.45% |
| α-amylase mutant 36 | 7.90% |
| α-amylase mutant 37 | 8.11% |
| α-amylase mutant 38 | 9.00% |
| α-amylase mutant 39 | 7.89% |
| α-amylase mutant 40 | 8.07% |
| α-amylase mutant 41 | 9.13% |
| α-amylase mutant 42 | 8.55% |
| α-amylase mutant 43 | 8.36% |

From Table 7 and FIG. 6, it can be seen that when using the enzyme solutions of α-amylase mutants 1-9, 11-13, 15-23, 25-30, 33-35, 37-38, and 41-43 for the liquefaction experiment with corn starch, their liquefaction effects were superior to that of the parental α-amylase. Specifically, the reducing sugar content after liquefaction was higher than that obtained with the parental α-amylase.

### 7. Liquefaction Experiment of Cornmeal for Ethanol Production

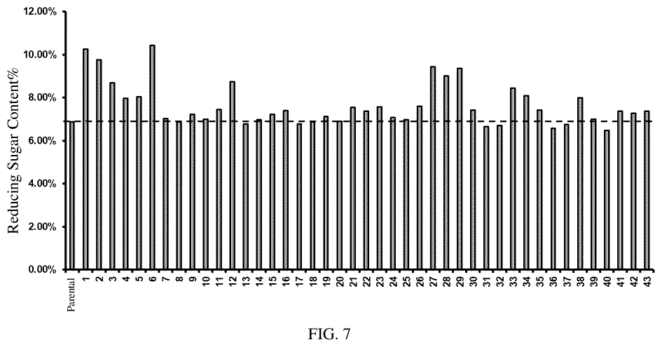

A corn slurry with a dry solids (DS) concentration of 25% was prepared, and its pH was adjusted to 4.8 using dilute sulfuric acid. A total of 500 g of the corn slurry was added to the liquefaction tank, and 15 U/g of the parental α-amylase and each α-amylase mutant enzyme solution were added separately. The reaction process was set as follows: 60° C. for 30 min, followed by 88° C. for 120 min, and finally 30° C. for 30 min. After the reaction, the reducing sugar content was measured, and the results are shown in Table 8 and FIG. 7.

TABLE 8

| | Reducing sugar |
|---|---|
| Group | content |
| Parental α-amylase | 6.87% |
| α-amylase mutant 1 | 10.25% |
| α-amylase mutant 2 | 9.76% |
| α-amylase mutant 3 | 8.68% |
| α-amylase mutant 4 | 7.97% |
| α-amylase mutant 5 | 8.04% |
| α-amylase mutant 6 | 10.43% |
| α-amylase mutant 7 | 7.02% |
| α-amylase mutant 8 | 6.88% |
| α-amylase mutant 9 | 7.22% |
| α-amylase mutant 10 | 6.99% |
| α-amylase mutant 11 | 7.45% |
| α-amylase mutant 12 | 8.74% |
| α-amylase mutant 13 | 6.77% |
| α-amylase mutant 14 | 6.98% |
| α-amylase mutant 15 | 7.22% |
| α-amylase mutant 16 | 7.40% |
| α-amylase mutant 17 | 6.78% |
| α-amylase mutant 18 | 6.87% |
| α-amylase mutant 19 | 7.11% |
| α-amylase mutant 20 | 6.90% |
| α-amylase mutant 21 | 7.55% |
| α-amylase mutant 22 | 7.38% |
| α-amylase mutant 23 | 7.56% |
| α-amylase mutant 24 | 7.06% |
| α-amylase mutant 25 | 6.97% |
| α-amylase mutant 26 | 7.60% |
| α-amylase mutant 27 | 9.44% |
| α-amylase mutant 28 | 9.00% |
| α-amylase mutant 29 | 9.35% |
| α-amylase mutant 30 | 7.43% |
| α-amylase mutant 31 | 6.65% |
| α-amylase mutant 32 | 6.70% |
| α-amylase mutant 33 | 8.43% |
| α-amylase mutant 34 | 8.10% |
| α-amylase mutant 35 | 7.43% |
| α-amylase mutant 36 | 6.58% |
| α-amylase mutant 37 | 6.76% |
| α-amylase mutant 38 | 7.98% |
| α-amylase mutant 39 | 6.99% |
| α-amylase mutant 40 | 6.48% |
| α-amylase mutant 41 | 7.38% |
| α-amylase mutant 42 | 7.26% |
| α-amylase mutant 43 | 7.37% |

From Table 8 and FIG. 7, it can be seen that when using the enzyme solutions of α-amylase mutants 1-12, 14-16, 19-30, 33-35, 38-39, and 41-43 for the liquefaction with com slurry, their liquefaction effects were superior to that of the parental α-amylase. Specifically, the reducing sugar content after liquefaction was higher than that obtained with the parental α-amylase.

In summary, compared to the parental α-amylase, the α-amylase mutants provided in the embodiments of the present disclosure exhibit at least one advantage in terms of heat resistance or acid resistance, and most of the α-amylase mutants possess both excellent acid resistance and heat resistance, as well as higher catalytic activity for starch hydrolysis, which can better meet the needs of starch-based deep processing industries such as sugar making, brewing, alcohol production, and organic acid production.

The above-mentioned embodiments merely represent several embodiments of the present disclosure, and their descriptions are relatively specific and detailed, but they should not be construed as limiting the scope of the present disclosure. It should be noted that, for those of ordinary skill in the art, several modifications and improvements can be made without departing from the concept of the present disclosure, and these all belong to the protection scope of the present disclosure. Therefore, the scope of protection of the patent of the present disclosure should be determined by the appended claims.

What is claimed is:

1. An α-amylase mutant, wherein compared with a parental α-amylase with an amino acid sequence shown in SEQ ID NO: 1, the α-amylase mutant has a disulfide bond site pair mutation, and the disulfide bond site pair has the following characteristics:
i. the disulfide bond site pair is not within 5 Å range of an active site of the α-amylase mutant;
ii. a difference value in amino acid site numbers is greater than 10;
iii. a distance between SG atoms of two cysteines forming a disulfide bond is within 5 Å; and
iv. a Chi3 angle is 60°<Chi3<120° or −60°>Chi3>−120°.

2. The α-amylase mutant according to claim 1, wherein the Chi3 angle of the disulfide bond site pair is 65°<Chi3<120° or the Chi3 angle is −65°>Chi3>−120°.

3. The α-amylase mutant according to claim 1, wherein compared with the parental α-amylase with the amino acid sequence shown in SEQ ID NO: 1, the α-amylase mutant has improved characteristics, wherein the improved characteristics include an increased pH stability and/or an increased thermostability.

4. The α-amylase mutant according to claim 3, wherein the pH stability includes acid resistance after being stored at pH 4.0 for 2 h.

5. The α-amylase mutant according to claim 3, wherein the increased thermostability includes increased stability at 70-99° C., and/or enhanced stability after being diluted 10-fold with a buffer at pH 4.5 and subjected to heat treatment at 95° C.

6. The α-amylase mutant according to claim 1, wherein compared with the parental α-amylase with the amino acid sequence shown in SEQ ID NO: 1, the α-amylase mutant has the disulfide bond site pair mutation as shown in any one of (1) to (43):
(1) Q97C, D227C; (2) P346C, K381C; (3) A2C, E416C; (4) E120C, S131C; (5) N127C, G192C; (6) D184C, K238C; (7) P243C, T281C; (8) Y266C, N291C; (9) V325C, A348C; (10) W183C, D195C; (11) G66C, T76C; (12) Y99C, G228C; (13) Q356C, Q397C; (14) T140C, A200C; (15) M204C, F241C; (16) V221C, T253C; (17) D18C, S53C; (18) Y60C, G109C; (19) Y394C, V414C; (20) W183C, N193C; (21) S297C, D341C; (22) D286C, L313C; (23) G301C, D428C; (24) A110C, W139C; (25) E412C, G439C; (26) V116C, A138C; (27) A27C, A90C; (28) G108C, Y199C; (29) S433C, K469C; (30) E120C, R174C; (31) P384C, T451C; (32) T410C, M436C; (33) G20C, G79C; (34) G408C, L425C; (35) F12C, P44C; (36) L22C, Y78C; (37) L289C, T312C; (38) T410C, A423C; (39) D286C, T323C; (40) L450C, V479C; (41) V116C, W158C; (42) M314C, E357C; (43) Q10C, W42C.

7. A nucleic acid molecule, encoding the α-amylase mutant according to claim 1.

8. A recombinant expression vector, comprising the nucleic acid molecule according to claim 7.

9. The recombinant expression vector according to claim 8, wherein a vector of the recombinant expression vector is a plasmid; and the plasmid includes a pBE-S plasmid.

10. A recombinant bacterium, comprising the nucleic acid molecule according to claim 7.

11. The recombinant bacterium according to claim 10, wherein the recombinant bacterium is selected from Escherichia coli or Bacillus.

12. An enzyme-containing composition, comprising the α-amylase mutant according to claim 1.

13. A use of the α-amylase mutant according to claim 1 in the production of syrup and/or alcohol.


SEQUENCE LISTING (2 sequences)

SEQ ID NO: 1 - protein, 481 aa, synthetic construct
AAPFNGTMMQYFEWYLPDDGTLWTKVANEANNLSSLGINALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWDHFDGVDWDESRKLSRIYKFRGKAWDWEVDTEFGNYDYLMYADLDMDHPEVVTELKNWGKWYVNTTNIDGFRLDAVKHIKFQFFPDWLSYVRSQTGKPLFTVGEYWSYDINKLHNYITKTDGTMSLFDAPLHNKFYTASKSGGAFDMRTLMTNTLMKDQPHLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSIYVQR

SEQ ID NO: 2 - protein, 513 aa, Geobacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGKAWDWEVDTEFGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP